(19) United States
(12) Patent Application Publication
(10) Pub. No.: US 2011/0071079 A1
(43) Pub. Date: Mar. 24, 2011

(54) SELF-ASSEMBLING POLY(DIOL CITRATES)-PROTEIN HYDROGELS

(76) Inventors: Guillermo Ameer, Chicago, IL (US); Haichao Zhao, Evanston, IL (US)

(21) Appl. No.: 12/887,251

(22) Filed: Sep. 21, 2010

Related U.S. Application Data
(60) Provisional application No. 61/244,264, filed on Sep. 21, 2009.

(51) Int. Cl. A61K 38/16; A61K 38/38; A61K 38/39

(52) U.S. Cl. 514/9.3; 514/1.1; 514/15.2; 514/15.3; 514/13.5

(57) ABSTRACT

The present invention can be directed to poly(diol-citrate)-based copolymers, compositions thereof comprising protein components and methods of use and assembly.

This application claims priority benefit from application Ser. No. 61/244,264 filed Sep. 21, 2009, the entirety of which is incorporated herein by reference.

### BACKGROUND OF THE INVENTION

Hydrogels are cross-linked, three-dimensional, hydrophilic polymer networks that can swell but not dissolve in water. Usually, hydrogels are prepared through covalent bonding such as crosslinking copolymerization, crosslinking of polymer precursors and polymer-polymer reactions, or through non-covalent interactions such as hydrogen bonding, electrostatic interaction, and hydrophobic effects. Hydrogels derived from biological macromolecules such as proteins and polysaccharides are of great interest since their components are analogous to those in living cells, but a major drawback of biological macromolecule-derived hydrogels is the limited control over their physical and biodegradation properties. Hybrid hydrogels formed by conjugation of natural biological macromolecules and synthetic polymers may lead to novel materials with properties superior to those of the individual components. Generally, one of the components of a hybrid hydrogel is a hydrophilic synthetic polymer, and the other is a biological macromolecule conjugated to the polymer by chemical or physical cross-linking.

### SUMMARY OF THE INVENTION

This invention can relate to the design, synthesis, and characterization of novel poly(diol citrates) that when mixed with protein can induce the formation of a hydrogel.

In part, this invention can be directed to a composition comprising the admixture of a polymer comprising a condensation product of citric acid and a diol; and a protein component.

In part, this invention can also be directed to a composition comprising a gelation product of a protein component and a polymer component comprising a repeating unit of a formula

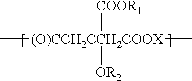

wherein X can be selected from C₂- about C₂₀ alkyl oxide moieties and a poly(alkylene oxide) moiety; and R₁ and R₂ can be independently selected from H and cross-linking components, such a protein component as can be present and not covalently bonded to such a polymer component. Such a polymer component and/or a repeating unit thereof can be at least partially deprotonated and can comprise an acid salt and a corresponding counter ion. Without limitation, such a polymer can comprise an alkali or alkaline earth metal salt thereof.

In part, this invention can also be directed to a method of using a protein component to gel an aqueous polymer composition. Such a method can comprise providing an aqueous medium comprising a polymer component comprising a condensation product of citric acid and a diol; and admixing a protein component with such an aqueous medium, such a protein component as can be in an amount at least partially sufficient to gel said aqueous medium. Without limitation, gelation can be determined, as described herein.

Another non-limiting feature of this invention can relate to the preparation of injectable two component hydrogels—and/or a kit corresponding thereto—through gelation of, for instance, citric acid-based water soluble poly(diol citrates) and proteins such as bovine serum albumin and fibrinogen.

Hydrogel preparation is easy, and the materials are inexpensive. The hydrogels can be formed within minutes to hours, depending on the composition, temperature and pH. Representative of various other compositions and methods of this invention, such poly(diol citrate)-protein hydrogels can have wide application in the food science industry and for use in drug delivery, wound healing, cell encapsulation, and tissue engineering.

### BRIEF DESCRIPTION OF THE DRAWINGS

FIG. 1 shows a reaction scheme used to prepare the poly (diol citrates).

FIG. 2 shows a reaction scheme used to prepare iminodiacetate-containing poly(diol citrates).

FIG. 3 is an interaction phase diagram of poly(1)-BSA system showing selected points characterized as solution or gel solely on the basis of their fluidity.

FIG. 4 is an interaction phase diagram of poly(2)-BSA system showing selected points characterized as solution or gel solely on the basis of their fluidity.

FIG. 5 is an interaction phase diagram of poly(3)-BSA system showing selected points characterized as solution or gel solely on the basis of their fluidity.

FIG. 6 is an interaction phase diagram of poly(4)-BSA system showing selected points characterized as solution or gel solely on the basis of their fluidity.

FIG. 7 shows digital pictures of typical polymer-protein hydrogels'(a) poly(2)-BSA hydrogel, (b) poly(3)-BSA hydrogel.

FIG. 8 shows typical microstructure of hydrogel after freeze drying (a) poly(4)-BSA with 4 wt % BSA and 16 wt % poly(3), (b) poly(4)-BSA hydrogel with 13.7 wt % BSA and 6.3 wt % polymer, (c) poly(1)-BSA hydrogel with 10 wt % poly(1) and 10 wt % BSA, (d) poly(1)-fibrinogen hydrogel with 16% wt poly(1) and 4 wt % of fibrinogen.

### DETAILED DESCRIPTION OF CERTAIN EMBODIMENTS

The interactions between proteins and macromolecules have attracted much attention in a variety of contexts within protein purification, cosmetic and pharmaceutical applications, food technology and biotechnology. Certain embodiments of this invention can be illustrated with the preparation of hybrid hydrogel by combining water soluble poly(diol citrates) with protein domains by means of non-covalent bonding or interaction. Water soluble poly(diol citrates) can be synthesized by condensation of citric acid and diols in mild conditions (140-160° C., 1-3 hr) without using any catalyst. A corresponding hydrogel can be formed by mixing poly(diol citrates) with proteins from minutes to hours, which depended on the composition of poly(diol citrates)/protein composition, sorts of proteins, pH, temperature, water content, and salts concentration.

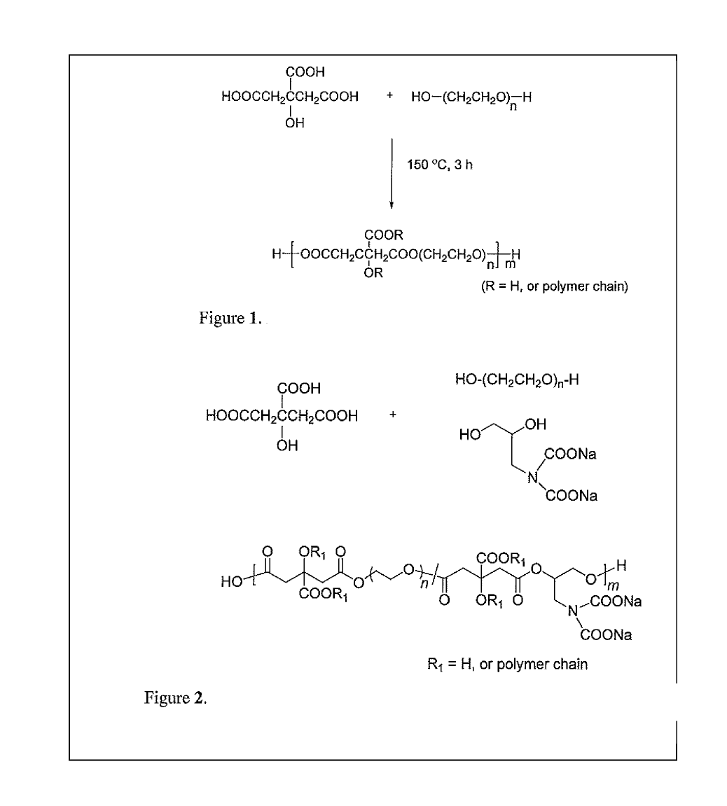

For instance, as illustrated through the referenced figures and tables, two types of water-soluble poly(diol citrates) were developed as examples for preparing the hybrid hydrogels. One type of poly(diol citrate) was prepared by polycondensation of citric acid and poly(ethylene glycol) (FIG. 1). The other type of poly(diol citrate) was prepared by polycondensation of citric acid, poly(ethylene glycol) and iminodiacetic acid-bearing diol. (FIG. 2.) Water soluble poly(diol citrates) containing hydroxyproline have also been synthesized.

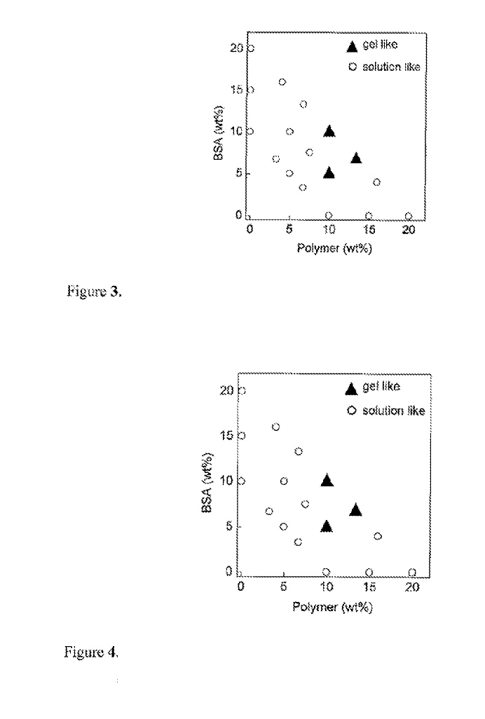

Poly(1)-BSA and poly(2)-BSA with the composition of 10/10, 13.3/6.7, and 10/5 weight percent formed hydrogels after incubating at 37° C. for 24 hr as shown in FIGS. 3 and 4.

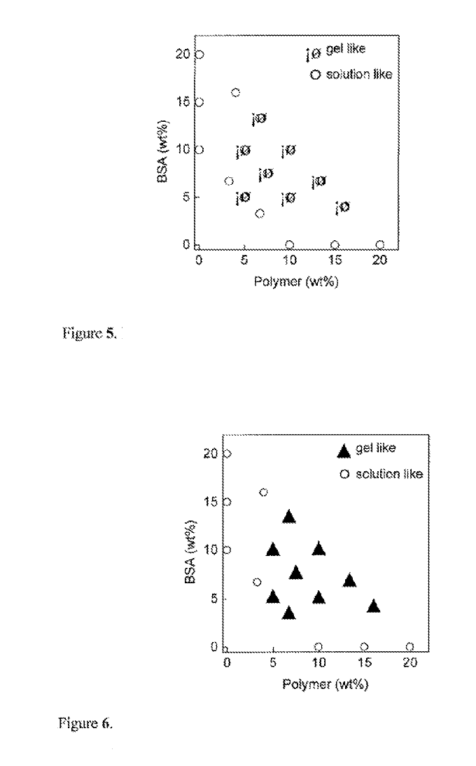

Poly(3) and poly(4) are imidiodiacetic acid containing polymers; they could form hydrogels with BSA with 90 weight percent water (FIGS. 5 and 6).

Table 1 summarizes the formation of solution or gel solely on the basis of the fluidity of poly(1)/fibrinogen after incubating at 37° C. for 24 hr. Typically, addition of only 1 weight percent of fibrinogen to 19 weight percent poly(1) in water could form hydrogel.

Tables 3 and 4 show the effect of pH on the gelation of poly(1)-BSA and poly(1)-fibrinogen, respectively. The hydrogel could be formed in at native pH or at lower pH values.

The salt effects on the gelation of poly(1)-BSA hydrogels were also examined (Table 4 and 5). The addition of NaCl to the gelation system could harden the gel, and the addition of CaCl₂ to the gelation system could accelerate the gel formation and change the mechanical properties.

TABLE 1
The formation of solution or gel solely on the basis of the fluidity
poly(1)/fibrinogen after incubating at 37° C. for 24 hr.

| | Sample No. | | | | | | | | |
|---|---|---|---|---|---|---|---|---|---|
| | 1 | 2 | 3 | 4 | 5 | 6 | 7 | 8 | 9 |
| Polymer (wt %) | 19 | 18 | 16 | 12 | 8 | 4 | 8 | 0 | 16 |
| Fibrinogen (wt %) | 1 | 2 | 4 | 8 | 12 | 16 | 2 | 4 | 0 |
| gel | + | + | + | +/− | − | − | + | − | − |

TABLE 2
pH effect on the gelation of poly(1)-BSA hydrogel¹

| | Sample No. | | | | | |
|---|---|---|---|---|---|---|
| | 1 | 2 | 3 | 4 | 5 | 6 |
| pH | 1.06 | 1.67 | 2.47 | 3.75 (native) | 4.86 | 5.87 |
| gel | + | + | + | + | − | − |

¹Condition: 10 wt % BSA, 10 wt % polymer incubated at 45° C. for 24 hr.

TABLE 3
pH effect on the gelation of poly(1)/fibrinogen hydrogel¹

| | Sample No. | | |
|---|---|---|---|
| | 1 | 2 | 3 |
| pH | 2.07 | 3.08 (native) | ~4 |
| gel | + | + | − |

¹Condition: 2% fibrinogen, 8% prepolymer, room temperature, 24 hr

TABLE 4
monovalent salt effects on the gelation of poly(1)/BSA hydrogel¹

| | No. | | | | |
|---|---|---|---|---|---|
| | 1 | 2 | 3 | 4 | 5 |
| NaCl (M) | 0.1 | 0.05 | 0.025 | 0.0125 | 0 |
| gel | + | + | + | + | + |

Rigid<-----------------------> Soft
¹Condition: 10 wt % BSA, 10 wt % polymer incubated at 45° C. for 24 hr.

TABLE 5
divalent salt effects on the gelation of poly(1)/BSA hydrogel

| | | | | | |
|---|---|---|---|---|---|
| CaCl₂ (M) | 0.1 | 0.05 | 0.025 | 0.0125 | 0 |
| gel | + | + | + | + | + |

| | |
|---|---|
| appearance | Opaque<-----------> Transparent |
| gelation time | Fast<---------------> Slow |

¹Condition: 10 wt % BSA, 10 wt % polymer incubated at 45° C. for 24 hr.

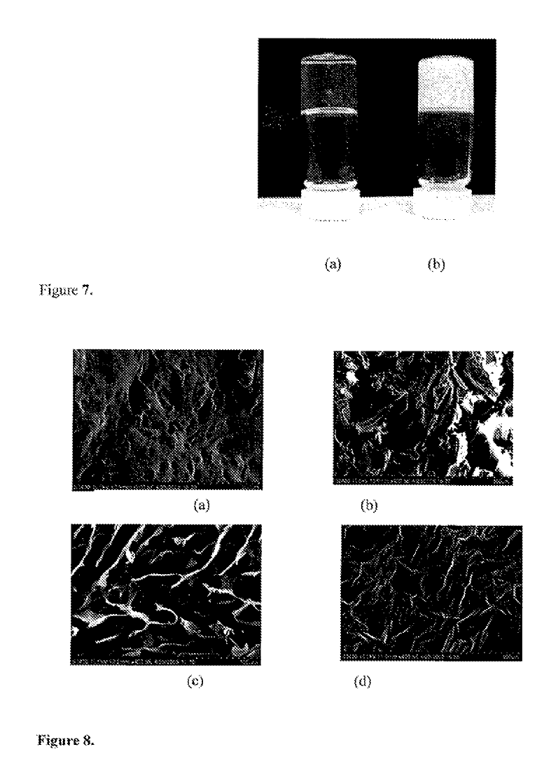

FIG. 7 shows digital images of the hydrogels. The formed hydrogels are transparent or opaque depending on the composition of polymer and protein and the presence or absence of divalent cations.

FIG. 8 provides digital images of a hydrogel microstructure after freeze drying. For polymer-BSA-based hydrogels, the freeze dried hydrogel showed porous structure, while for fibrinogen-based hydrogels, the porous structure shows collapsed pores after freeze drying.

With respect to one or more of the preceding embodiments, this invention can provide a water-soluble, biodegradable, polyelectrolyte composition which includes a citric acid-poly(ethylene glycol) (PEG) segment. A representative composition is in a 1:1 molar ratio (citric acid to PEG). Such a composition can also include a second diol or polyol that has a functional group such as iminodiacetic acid (IDA), an aminoacid, or a peptide. The second diol or polyol may be present in a mole ratio as high as 1:1 with respect to the poly(ethylene glycol) component of the polyelectrolyte. Up to half of the PEG content can be replaced with an aminoacid and/or a compound containing a carboxyl and hydroxyl group. In certain embodiments, the amino acid is hydroxyproline. Regardless, a biodegradable gel can comprise a water soluble poly(diol citrate) and a protein. Examples of a protein include albumin, fibrinogen, fibronectin, hemoglobin, and laminin.

From another perspective, this invention can be directed to a method to prepare biodegradable water-soluble poly(diol citrates), such as by polycondensation of citric acid with diols, triols, hydroxyl acids, and/or amino acids, etc. under mild conditions without using a catalyst. To prepare biodegradable poly(diol citrates)-protein hydrogels, a polyelectrolyte such as that described above is mixed with any protein. Optionally, calcium or any divalent cation (or salt thereof such as ZnCl₂, CaCl₂, CuCl₂) is added to the solution to stabilize or speed up the formation of the hydrogel.

Regardless, such a composition and/or method can be used in conjunction with a method to remove cations from a solution; a method to deliver proteins; a method to encapsulate cells whereby the encapsulation or entrapment vehicle is such a gel; a method to deliver drugs from the gel, whereby the drug is a component of the gel or the product of a reaction within the gel; a method to obtain local delivery of nitric oxide from the gel, whereby nitric oxide is generated from the decomposition of diazeniumdiolates formed via the hydroxyproline component of the gel; a method to obtain local delivery of nitric oxide from the gel, whereby nitric oxide is generated from the decomposition of an s-nitroso group such as S-nitroso albumin; and a method to obtain selective attachment of cells using gels, whereby these gels may incorporate cell adhesive or non-adhesive signals to form patterns within a gel or on surfaces.

More generally, without limitation, polymer components of the compositions of this invention can be of the sort described in co-pending patent application Ser. No. 10/945,354 (filed Sep. 20, 2004 and published on Mar. 24, 2005), application Ser. No. 12/586,365 (filed Sep. 21, 2009 and published on Mar. 25, 2010) and application Ser. No. 11/529,064 (filed Sep. 28, 2006 and published on Mar. 29, 2007), each of which is incorporated herein by reference in its entirety. Likewise, more generally, protein components of the present invention can include any food grade protein of plant, animal or microbial source acceptable for human consumption, including but not limited to the proteins described in U.S. Pat. Nos. 7,169,425 and 7,597,921; or protein biomolecules and/or therapeutic agents known in the art, including but not limited to those described in co-pending patent application Ser. No. 12/681,682 (filed Apr. 5, 2010 and published on Sep. 2, 2010) and the aforementioned '064 patent application, each of which is incorporated herein by reference in its entirety.

### EXAMPLES OF THE INVENTION

The following non-limiting examples and data illustrate various aspects and features relating to the compositions and/or methods of the present invention, including the assembly of various hydrogel compositions, as are available through the synthetic methodologies described herein. In comparison with the prior art, the present compositions and methods provide results and data which are surprising, unexpected and contrary thereto. While the utility of this invention is illustrated through the use of several compositions and polymers and protein components which can be used therewith, it will be understood by those skilled in the art that comparable results are obtainable with various other compositions, polymers and/or protein components, as are commensurate with the scope of this invention.

### Materials

Citric acid, poly(ethylene glycol) (Mw=400), tetraethylene glycol, glycidol, iminodiacetic acid and sodium hydroxide were purchased from Sigma-Aldrich, and were used without purification. Iminodiacetic acid-based diol was synthesized by reaction of glycidol and iminodiacetic acid. Specifically, iminodiacetic acid (300 mmol) and sodium hydroxide (600 mmol) were added into 300 ml distilled water, and then glycidol (300 mmol) was dripped into the mixture slowly. The reaction was conducted at 50° C. for 5 hours, followed by evaporation of the solvents. The crude product was dissolved in 100 ml distilled water and precipitated in a large amount of acetone, followed by drying in vacuum to a constant weight.

### Example 1

### Preparation of Poly(ethylene glycol) (Mw=400) and Citric Acid-Based Poly(diol citrate) (Poly(1))

The poly(1) was synthesized by condensation of citric acid, poly(ethylene glycol) diol. Typically, citric acid (300 mmol) and poly(ethylene glycol) (300 mmol) were added to a 500 ml round bottom flask. The mixture was polymerized at 150° C. for 3 hours to get the crude polymer. The crude polymer was dissolved in 100 ml EtOH and precipitated in a larger amount of ether, followed by drying in vacuum to a constant weight.

### Example 2

### Preparation of Tetraethylene Glycol and Citric Acid-Based Poly(diol citrate) (Poly(2))

Typically, citric acid (300 mmol) and poly(ethylene glycol) (300 mmol) were added to a 300 ml round bottom flask. The mixture was polymerized at 140° C. for 1.5 hours to get the crude polymer. The crude polymer was dissolved in 100 ml EtOH and precipitated in a large amount of ether, followed by drying in vacuum to a constant weight.

### Example 3

### Preparation of Poly(Diol Citrate) by Condensation of Citric Acid, Poly(ethylene glycol) (Mw=400) and Iminodiactic Acid-Base Diol (Poly(3))

Citric acid (200 mmol), poly(ethylene glycol) (120 mmol) were added to a 250 ml round bottom flask, and the mixture was polymerized at 150° C. for 1 hour; then iminodiacetic acid-containing diol (80 mmol) in 50 water was added to the flask slowly. The polymerization was conducted for another 1 hour to get the crude polymer. The crude polymer was dissolved in 100 ml water and was precipitated in a large amount of acetone, followed by drying in vacuum to a constant weight.

### Example 4

### Preparation of Poly(Diol Citrate) by Condensation of Citric Acid, Tetraethylene Glycol, and Iminodiacetic Acid-Based Diol (Poly(4))

Citric acid (300 mmol), tetraethylene glycol (180 mmol) and imino diacetic acid-containing diol (120 mmol) in 50 ml water were added to a 300 ml round bottom flask. The mixture was polymerized at 140° C. for 1.5 hours to get the crude polymer. The crude polymer was dissolved in 100 ml water and precipitated in acetone, followed by drying in vacuum to a constant weight.

### Example 5

### Preparation of Poly(diol citrate)-Protein Hydrogel

Typical example: (10 wt % BSA, 10 wt % poly(2)-based hydrogel): BSA, illustrating a representative protein component of the compositions of this invention, (300 mg) and poly(2)(300 ml) were dissolved in 2.4 g distilled water. The complex was shaken for a certain time to form a transparent solution, and then solution was incubated at 37° C. for 24 hour to get a hydrogel. Depending on the composition and temperature of the mixture, gels may form within 10 minutes, 30 minutes, 3 hours, or 24 hours. Various other protein components of the sort described or referenced herein can be utilized, accordingly, regardless of polymer identity.

### Example 6

### Preparation of Poly(1,8-Octanediol-co-citric acid) (POC)

In a typical experiment, 19.212 g citric acid and 14.623 g octanediol were added to a 250 mL three-neck round-bottom flask, fitted with an inlet adapter and an outlet adapter. The mixture was melted within 15 min by stirring at 160-165° C. in silicon oil bath, and then the temperature of the system was lowered to 140° C. The mixture was stirred for another 1 hr at 140° C. to get the crude polymer. Nitrogen was vented throughout the above procedures.

### Example 7

### Synthesis of Poly(1,6-hexanediol-co-citric acid) (PHC)

In a typical experiment, 19.212 g citric acid and 11.817 g 1,6-hexanediol were added to a 250 ml three-neck round-bottom flask, fitted with an inlet adapter and an outlet adapter. The mixture was melted within 15 min by stirring at 160-165° C. in a silicon oil bath, and then the temperature of the system was lowered to 120° C. The mixture was stirred for half an hour at 120° C. to get the crude polymer. Nitrogen was vented throughout the above procedures.

### Example 8

### Synthesis of Poly(1,10-decanediol-co-citric acid) (PDC)

In a typical experiment, 19.212 g citric acid and 17.428 g 1,10-decanediol were added to a 250 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for half an hour at 120° C. to get the crude polymer. Nitrogen was vented throughout the above procedures.

### Example 9

### Synthesis of Poly(1,12-dodecanediol-co-citric acid) PDDC

In a typical experiment, 19.212 g citric acid and 20.234 g 1,12-dodecanediol were added to a 250 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for half an hour at 120° C. to get the crude polymer. Nitrogen was vented throughout the above procedures.

### Example 10

### Synthesis of Poly(1,8-octanediol-co-citric acid-co-glycerol)

In a typical experiment, 23.0544 g citric acid, 16.5154 g 1,8-octanediol and 0.2167 g glycerol were added to a 250 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for another hour at 140° C. to get the crude polymer. Nitrogen was vented throughout the above procedures.

### Example 11

### Synthesis of Poly(1,8-octanediol-citric acid-co-polyethylene oxide)

In a typical experiment, 38.424 g citric acid, 14.623 g 1,8-octanediol and 40 g polyethylene oxide with molecular weight 400 (PEO400) (100 g PEO1000 and 200 g PEO2000 respectively) (molar ratio: citric acid/1,8-octanediol/PE0400=1/0.5/0.5) were added to a 250 ml or 500 ml three-neck round-bottom flask, fitted with an inlet adapter and an outlet adapter. The mixture was melted within 15 min by stirring at 160-165° C. in silicon oil bath, and then the temperature of the system was lowered to 135° C. The mixture was stirred for 2 hours at 135° C. to get the crude polymer. Nitrogen was vented throughout the above procedures.

### Example 12

### Synthesis of Poly(1,12-dodecanediol-citric acid-co-polyethylene oxide)

in a typical experiment, 38.424 g citric acid, 20.234 g 1,12-dodecanediol and 40 g polyethylene oxide with molecular weight 400 (PE0400)(100 g PEO1000 and 200 g PEO2000 respectively) (molar ratio: citric acid/1,8-octanediol/PEO400=1/0.5/0.5) were added to a 250 ml- or 500 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for half an hour at 120° C. to get the crude polymer. Nitrogen was vented throughout the above procedures.

### Example 13

### Synthesis of Poly(1,12-dodecanediol-citric acid-co-N-methyldiethanoamine) PDDCM

In a typical experiment, 38.424 g citric acid, 36.421 g 1,12-dodecanediol and 2.3832 g N-methyldiethanoamine (MDEA) (molar ratio: citric acid/1,8-octanediol/MDEA=1/0.90/0.10) were added to a 250 ml or 500 ml three-neck round-bottom flask, fitted with an inlet adapter and an outlet adapter. The mixture was melted within 15 min by stirring at 160-165° C. in a silicon oil bath, and then the temperature of the system was lowered to 120° C. The mixture was stirred for half an hour at 120° C. to get the crude polymer. Nitrogen was vented throughout the above procedures.

1. A composition comprising the admixture of a polymer comprising a condensation product of citric acid and a diol; and a protein component.

2. The composition of claim 1 wherein said diol is a glycol.

3. The composition of claim w wherein said diol is a poly(alkylene glycol).

4. The composition of claim 1 wherein said polymer comprises a condensation product of citric acid, a diol and a polyol.

5. The composition of claim 4 where said diol is independently selected from a poly(alkylene glycol) and a C₂- about C₂₀ alkanediol; and said polyol is independently selected from glycerol, a dialkanolamine, a poly(alkylene glycol) and a condensation product of glycerol and an amine.

6. The composition of claim 5 wherein said polymer is the polycondensation product of citric acid, poly(ethylene glycol) and an alkanediol

7. The composition of claim 1 wherein said protein component is selected from food grade plant and animal proteins, and combinations of said proteins.

8. The composition of claim 7 wherein said protein component comprises a protein therapeutic agent.

9. The composition of claim 1 wherein said protein component is selected from an albumin, a fibrinogen, a fibronectin, a hemoglobin, and a laminin.

10. The composition of claim 1 in an aqueous medium, said protein component in an amount at least partially sufficient to gel said composition.

11. The composition of claim 1 in an aqueous medium, said composition comprising a cross-linking component selected from divalent cationic components, hydroxyproline and combinations of said components.

12. A composition comprising a gelation product of a protein component and a polymer component comprising a repeating unit of a formula 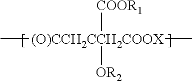
wherein X is selected from C₂- about C₂₀ alkyl oxide moieties and a poly(alkylene oxide) moiety; and R₁ and R₂ are independently selected from H and cross-linking components, said protein component not covalently bonded to said polymer component.

13. The composition of claim 12 wherein X is a C₆-C₁₄ alkyl oxide moiety.

14. The composition of claim 12 wherein X is a poly(ethylene oxide) moiety.

15. The composition of claim 14 comprising the condensation product of citric acid and a C₆-C₁₄ alkanediol.

16. The composition of claim 12 wherein each said linking component is independently selected from C₂- about C₂₀ alkanediols, hydroxyproline and polymeric components comprising said repeating unit.

17. The composition of claim 12 wherein said protein component is about 0.05 weight percent to about 15 weight percent of said composition.

18. A method of using a protein component to gel an aqueous polymer composition, said method comprising:
providing an aqueous medium comprising a polymer component comprising a condensation product of citric acid and a diol; and
admixing a protein component with said aqueous medium, said protein component in an amount at least partially sufficient to gel said aqueous medium.

19. The method of claim 18 wherein said protein component is about 0.05 weight percent to about 15 weight percent of said composition.

20. The method of claim 18 wherein said admixture is heated for a time and at a temperature to affect said gelation.
